(19) United States
(12) Patent Application Publication
(10) Pub. No.: US 2004/0127403 A1
(43) Pub. Date: Jul. 1, 2004

(54) Methods for treating and preventing Gram-positive bacteremias

(76) Inventors: Francesco Parenti, Lainate (IT); Henry Fuchs, San Francisco, CA (US); Timothy S. Leach, Groton, MA (US)

Correspondence Address: CLARK & ELBING LLP, 101 FEDERAL STREET, BOSTON, MA, 02110, US

(21) Appl. No.: 10/655,185

(22) Filed: Sep. 4, 2003

Related U.S. Application Data
(60) Provisional application No. 60/408,596, filed on Sep. 6, 2002.
(60) Provisional application No. 60/419,117, filed on Oct. 18, 2002.

(51) Int. Cl. A61K 38/13; A61K 31/4745

(52) U.S. Cl. 514/11; 514/291; 424/145.1

(57) ABSTRACT

The present invention provides methods and compositions useful for preventing a bacteremia by administering ramoplanin to decolonize the intestinal tract of a patient. Also disclosed are methods for treating bacteremias using combination therapy directed both toward treating the infection as well as decolonizing the intestinal tract of the patient. The invention is particularly useful against antibiotic-resistant Gram-positive bacteria, such as vancomycin-resistant Enterococcus (VRE), methicillin-resistant Staphylococcus aureus (MRSA), vancomycin-resistant Staphylococcus aureus (VRSA), glycopeptide intermediary susceptible Staphylococcus aureus (GISA), and coagulase-negative staphylococci.

### CROSS REFERENCE TO RELATED APPLICATIONS

This application claims benefit of the filing date of U.S. Provisional Application No. 60/408,596 (filed Sep. 6, 2002) and No. 60/419,177 (filed Oct. 18, 2002), hereby incorporated by reference.

### FIELD OF THE INVENTION

This invention relates to the field of mammalian bacterial infections.

### BACKGROUND OF THE INVENTION

Gram-positive bacteria are becoming an important cause of nosocomial infection. The most common pathogenic isolates in hospitals include Enterococcus spp., Staphylococcus aureus, coagulase-negative staphylococci (Principles and Practice of Infectious Diseases, 4th ed. Mandell G L, Bennett J E, Dolin R, ed. Churchill Livingstone, New York 1995), and Streptococcus pneumoniae, many strains of which are resistant to one or more antibiotics.

Enterococcus spp. are part of the normal gut flora in humans. Of the more than seventeen enterococcal species, only E. faecalis and E. faecium commonly colonize and infect humans in detectable numbers (E. faecalis is isolated from approximately 80% of human infections, and E. faecium from most of the rest). Enterococci account for approximately 25,000 cases of bacteremia annually in the United States, with most infections occurring in hospitals. Attributable mortality due to enterococcal infection has also been difficult to ascertain because severe comorbid illnesses are common; however, enterococcal sepsis is implicated in 7% to 50% of fatal cases.

Vancomycin-resistant enterococcus (VRE) spp. are becoming increasingly common in hospital settings. In the first half of 1999, 25.9% of entercoccal isolates from Intensive Care Units were vancomycin-resistant; an increase from 16.6% in 1996 and from 0.4% in 1989. VRE are also commonly resistant to many other commercial antibiotics, including beta-lactams and aminoglycosides. Thus, patients who are immunocompromised or those having a prolonged hospital stay are at increased risk for acquiring a VRE infection. Several case-control and historical cohort studies show that death risk associated with antibiotic-resistant enterococcal bacteremia is several fold higher than death risk associated with susceptible enterococcal bacteremia.

The problem of antibiotic resistance is not unique to Enterococcus spp. Strains of many other potentially pathogenic Gram-positive bacteria displaying antibiotic resistance have been isolated including methicillin-resistant Staphylococcus aureus (MRSA), vancomycin-resistant Staphylococcus aureus (VRSA), glycopeptide intermediate-susceptible Staphylococcus aureus (GISA), vancomycin-resistant MRSA (VR-MRSA) and penicillin-resistant Streptococcus pneumoniae (PRSP). Like VRE, therapeutic options for treating infections by these organisms are limited.

Resistance transfer is another complicating factor in the management of antibiotic-resistant infections. Enterococcus, for example, exhibits at least three phenotypes of vancomycin resistance: VanA—high level resistance to vancomycin and teicoplanin, VanB—moderate level resistance to vancomycin but susceptibility to teicoplanin, and VanC—low level resistance to vancomycin but susceptibility to teicoplanin. Vancomycin resistance can transfer from VRE to other Gram-positive bacteria, including S. aureus, in vitro. Therefore, the presence of VRE in a hospital poses not just the risk of VRE infections but also of continuing evolution of resistance, possibly involving more virulent organisms.

Despite the development of a plethora of new antibiotics, there is a need for new methods for treating or preventing bacteremia caused by resistant gastrointestinal bacterial flora and other Gram-positive bacteria such as VRE.

### SUMMARY OF THE INVENTION

We have discovered that blood infections by Gram-positive bacteria may be prevented in high risk patients by substantially decolonizing the intestinal tracts using an effective amount of orally administered ramoplanin. Likewise, decolonization therapy using ramoplanin may be used in conjunction with traditional antibiotic therapy for treating any patient diagnosed as having a Gram-positive bacteremia. The addition of the decolonization therapy using orally administered ramoplanin increases the effectiveness of the anti-bacteremia therapy by reducing or eliminating the gastrointestinal bacterial reservoir. Ramoplanin-induced decolonization also reduces the likelihood of a recurrence of the bacteremia.

Accordingly, in a first aspect, the invention provides a method for preventing a bacteremia in a high risk patient by: (a) identifying a high risk patient whose intestinal tract is colonized with Gram-positive bacteria, but who does not have a bacteremia caused by the bacteria; and (b) orally administering to the patient ramoplanin in an amount and for a duration sufficient to substantially decolonize the intestinal tract of the Gram-positive bacteria. In patients where the elevated risk of developing a bacteremia is a result of a medical procedure or treatment (e.g., antineoplastic or immunosuppressive therapy), it is preferable that ramoplanin therapy to substantially decolonize the intestinal tract begin at least 1 day, 2 days, 3 days, 4 days, 5 days, 6 days, 7 days, 10 days, or 14 days prior to the medical procedure or treatment. In one embodiment, decolonization proceeds concomitantly with the medical procedure. If desirable, the decolonization therapy may be continued for at least 1 day, 2 days, 3 days, 4 days, 5 days, 6 days, 7 days, 10 days, or 14 days subsequent to the medical procedure.

In a second aspect, the invention provides a method for treating a bacteremia in a patient, in which the bacteremia is caused by Gram-positive bacteria, by administering: (a) an bioavailable antibiotic in an amount and duration sufficient to treat the bacteremia; and (b) oral ramoplanin in an amount and for a duration sufficient to substantially decolonize the intestinal tract of the patient of the Gram-positive bacteria. The bioavailable antibiotic may be administered by any medically appropriate route including, for example, orally or by intravenous, intramuscular, or subcutaneous injection. Particularly useful bioavailable antibiotics suitable for treating systemic Gram-positive bacteremias include, for example, antibiotics belonging to the antibiotic families: beta lactams, aminoglycosides, flurorquinolones, glycopeptides, bacteriocins, type A lantibiotics, type B lantibiotics, liposidomycins, mureidomycins, alanoylcholines, quinolines, everninomycins, glycylcyclines, carbapenems, cephalosporins, streptogramins, oxazolidonones, tetracyclines, cyclothialidines, bioxalomycins, cationic peptides, or protegrins. Specifically, useful bioavailable antibiotics include, for example, almecillin, amdinocillin, amikacin, amoxicillin, amphomycin, amphotericin B, ampicillin, azacitidine, azaserine, azithromycin, azlocillin, aztreonam, bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, everninomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mupirocin, mycophenolic acid, nafcillin, natamycin, neomycin, netilmicin, niphimycin, nisin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, Pep5, RP 59500, and TD-6424.

Either of the foregoing aspects are particularly useful against Gram-positive bacteria such as Enterococcus spp. including E. faecium, E. faecalis, E. raffinosus, E. avium, E. hirae, E. gallinarum, E. casseliflavus, E. durans, E. malodoratus, E. mundtii, E. solitarius, and E. pseudoavium; Staphylococcus spp. including S. aureus, S. epidermidis, S. hominis, S. saprophyticus, S. hemolyticus, S. capitis, S. auricularis, S. lugdenis, S. warneri, S. saccharolyticus, S. caprae, S. pasteurii, S. schleiferi, S. xylosus, S. cohnii, and S. simulans; Streptococcus spp. including S. pyogenes, S. agalactiae, S. pneumoniae, S. bovis, S. aureus, and viridans Streptococci. Specifically, intestinal decolonization therapy using the methods and compositions of the present invention are effective for preventing or treating bacteremias caused by vancomycin-resistant Enterococcus spp. (VRE), methicillin- or glycopeptide-resistant Staphylococcus spp. (e.g., MRSA, VRSA, MRSE, GISA, or VR-MRSA), and penicillin-resistant Streptococcus spp. (e.g., PRSP). The methods and compositions of this invention are also useful for the prevention of bacteremia due to susceptible or multiply resistant species of Enterococcus, Staphylococcus, or Streptococcus.

In particularly useful embodiments of either of the foregoing aspects of the invention, ramoplanin is administered in a pharmaceutical formulation such that the ramoplanin is either non-absorbable or partially non-absorbable and retains antibacterial activity in the lumen of the intestinal tract of the patient. Ramoplanin may be administered at once, twice, three times, four time, or more frequently each day and the total daily dose may be 50 mg-2.0 g. Ramoplanin decolonization therapy may be administered for at least 7 days, at least 14 days, or longer if clinically indicated. Particularly useful administration regimens administer ramoplanin twice daily at a dosage of 100 mg-800 mg (total daily dose of 200 mg-1600 mg). Most desirably, ramoplanin is administered at a dosage of 200 mg-400 mg twice daily. Decolonization therapy according to either of the aspects of the invention is particularly useful against vancomycin-resistant enterococci, methicillin-resistant staphylococci, and any Gram-positive bacteria, but particularly enterococci and staphylococci, that are resistant to linezolid and/or quinupritin/dalfoprisin, By “high risk patient” is meant any patient that has an increased likelihood of developing a bacteremia caused by Gram-positive bacteria that have colonized the patient's gastrointestinal tract. High risk patients may have impaired immune function or have increased intestinal permeability. Impaired immune function in a patient may be iatrogenically-induced, or may result from a disease process or a genetic defect. Increased intestinal permeability may also result from iatrogenic causes, disease processes, or anatomic or physiologic defects.

Patients with malignancies are at high risk for bacteremia of gastrointestinal origin due to intestinal epithelial injury caused by antineoplastic therapy (e.g., chemotherapy and/or radiation therapy). Patients having a compromised barrier function of the intestinal tract are also at elevated risk for developing a bacteremia by bacteria that colonize their intestinal tract. Such conditions include patients receiving antineoplastic chemotherapy or radiation therapy, and those suffering antibiotic-induced colitis and those having, or at risk for developing, Crohn's disease, enteritis, colitis, or mucositis of the intestinal tract. Recipients of high dose chemotherapy (e.g., administration of corticosteroids, anti-thymocyte globulin, cyclosporin, and tacrolimus) followed by autologous or allogeneic hematopoietic stem cell transplant or bone marrow transplant or those diagnosed as having hematologic malignancies (e.g., leukemia) may require decolonization therapy before, during and after their treatment periods. Patients are at highest risk within (before or after) 14 days of receiving antineoplastic or immunosuppressive therapy.

Other high risk patients include patients diagnosed as having an illness requiring institutionalization in a hospital or other medical facility for at least five consecutive days, or in an intensive care unit for at least three consecutive days. At particular high risk are those patients institutionalized in facilities in which antibiotic-resistant Gram-positive bacteria (e.g., VRE and MRSA) are endemic.

High risk patients also include those that are diagnosed as having a human immunodeficiency virus (HIV) infection or acquired immunodeficiency syndrome (AIDS), chronic renal insufficiency, an autoimmune disorder (e.g., systemic lupus erythematosus, rheumatoid arthritis, scleroderma, dermatomyositis/polymyositis, Sjogren's syndrome, mixed connective tissue disorders, Behcet's syndrome, sarcoidosis, or vasculitides). Other factors that place patients at high risk are liver cirrhosis, alcoholism, malnutrition, extremes of age, diabetes, splenectomy, and sickle cell anemia.

Antibiotic therapies that cause a patient to be at “high risk” for developing an antibiotic-resistant Gram-positive bacteremia included prior or concomitant antibacterial therapy using vancomycin or an antibiotic with anaerobic bacterial activity (e.g., metronidazole).

By “patient” is meant a human in need of medical treatment. For the purposes of this invention, patients are typically institutionalized in a primary medical care facility such as a hospital or nursing home. However, antibiotic therapy for depopulating the intestinal tract of antibiotic-resistant Gram-positive bacteria can occur on an outpatient basis, upon discharge from a primary care facility, or can be prescribed by a physician (e.g., general practitioner) for home-care, not in association with a primary medical care facility.

By “antibiotic-resistant Gram-positive bacteria” is meant any Gram-positive bacteria that have reduced (partially or completely) susceptibility to one or more antibiotics. Antibiotic classes to which Gram-positive bacteria develop resistance include, for example, the penicillins (e.g., penicillin G, ampicillin, methicillin, oxacillin, and amoxicillin), the cephalosporins (e.g., cefazolin, cefuroxime, cefotaxime, and ceftriaxone, ceftazidime), the carbapenems (e.g., imipenem, ertapenem, and meropenem), the tetracyclines and glycylcylines (e.g., doxycycline, minocycline, tetracycline, and tigecycline), the aminoglycosides (e.g., amikacin, gentamicin, kanamycin, neomycin, streptomycin, and tobramycin), the macrolides (e.g., azithromycin, clarithromycin, and erythromycin), the quinolones and fluoroquinolones (e.g., gatifloxacin, moxifloxacin, sitafloxacin, ciprofloxacin, lomefloxacin, levofloxacin, and norfloxacin), the glycopeptides (e.g., vancomycin, teicoplanin, dalbavancin, and oritavancin), dihydrofolate reductase inhibitors (e.g., cotrimoxazole, trimethoprim, and fusidic acid), the streptogramins (e.g., synercid), the oxazolidinones (e.g., linezolid), the eveminomycins (e.g., everninonmycin), and the lipopeptides (e.g., daptomycin).

“Colonized” or “colonization,” as used herein, refers to a population of bacteria in the intestinal tract that is present in the intestinal tract, but does not cause disease. The population of the intestinal tract by normal intestinal flora, as described herein, is exemplary of what is meant by colonization.

By “substantially decolonize” is meant to reduce the population of competent target bacteria in the intestinal tract by at least two logs (base 10), as determined by the quantification of bacterial growth from a fecal sample, or to reduce the population to undetectable levels from a rectal swab. Each of these determinations can be performed using standard microbiological techniques, such as those that conform to the standards provided by the American Society for Microbiology (Manual of Clinical Microbiology (7^{th} ed.) eds. Murray P R, Barron E J, Pfaller M A, Tenover F C, and Yolken R H, 1999, American Society for Microbiology, Washington). Most desirably, complete decolonization results in a reduction of the competent population of target bacteria to levels that are undetectable by standard microbiological culture methods. Decolonization can also include the eradication or suppression of the bacteria.

By “decolonization therapy” is meant a regimen for administration of ramoplanin in an amount and duration sufficient to substantially decolonize the intestinal tract of a patient of Gram-positive bacteria (e.g., antibiotic-resistant Gram-positive bacteria). Preferably, decolonization therapy is provided prior to, during, and subsequent to the risk period for infection. Desirably, decolonization therapy is provided by maintaining the amount of ramoplanin in the stool of the patient at a concentration greater than the MIC for the bacteria that is the target of the therapy. Preferably, the antibiotic concentration in the stool is maintained at twice, three times, four times, five times, or higher multiple of the MIC for the target bacteria.

“Bacteremia” is defined as the presence of bacteria in the bloodstream of a patient, detectable using standard aerobic or anaerobic cultures of the blood or standard molecular biological techniques. A patient having a bacteremia may be symptomatic or asymptomatic.

“Non-absorbable” is defined as an antibiotic formulation which, when administered orally, has an absolute bioavailability of less than 10%, preferably less than 5%, more preferably less than 1%. A non-absorbable compound cannot be detected, as the parent compound or its biologically active metabolites, in the blood or urine of the patient following oral administration.

By “partially non-absorbable,” when referring to an antibiotic, is meant an antibiotic formulation which, when administered orally, results in an absolute bioavailability of between 10% and 90%.

By “bioavailable antibiotic” is meant any antibiotic suitable for treating a systemic (i.e., blood-borne) bacteremia. Bioavailable antibiotics may be bioavailable following oral administration (i.e., absorbed from the gastrointestinal tract in an amount sufficient to achieve a therapeutic concentration in the blood). Alternatively, parenteral administration (e.g., intravenous, intramuscular, and subcutaneous injection) of an antibiotic renders it bioavailable regardless of its oral bioavailability.

“Retains antibacterial activity” refers to a non-absorbable or partially non-absorbable antibiotic formulation which is at least 50%, 60%, 70%, 80%, 90%, 95%, or 99% bactericidal or bacteriostatic as a formulation of the same antibiotic that is more absorbable in the intestinal tract.

By “bioavailability” is meant the fraction (F) of the orally administered dose that reaches the systemic circulation (Oates J A, Wilkinson G R. Priniciples of drug therapy, In Harrison's Principle of Internal Medicine (14^{th}, ed.) 1998, McGraw Hill, New York.

### DETAILED DESCRIPTION

The present invention stems from our discovery that oral administration of ramoplanin, alone or in combination with another antibiotic, can prevent a Gram-positive bacteremia in a patient whose intestinal tract is colonized by such bacteria. Decolonization therapy is particularly effective for preventing bacteremias caused by antibiotic resistant Gram-positive bacteria (e.g., VRE, MRSA, VRSA, and GISA). Decolonization therapy may also be administered, in conjunction with systemic antibiotic therapy, to a patients diagnosed as having a bacteremia in order to prevent a re-infection from gastrointestinal bacterial reservoirs.

Patients at Risk for Developing a Bacteremia

Patients that are particularly vulnerable to blood-borne infection are those that are immunocompromised. Conditions that compromise the immune system include disorders and diseases such as malignancy, neutropenia, HIV infection or AIDS, or other viral or parasitic infections, chronic renal insufficiency, cirrhosis, alcoholism, extremes of age, connective tissue disorders, malnutrition, diabetes, splenectomy, sickle cell anemia, or concurrent administration of corticosteriods, immunosuppressants, or cytotoxic drugs. Patients with malignancies are also at high risk for bacteremia of gastrointestinal origin due to intestinal epithelial injury caused by chemotherapy and/or radiation therapy. Patients having a compromised barrier function of the intestinal tract are also at elevated risk for developing a bacteremia by bacteria that colonize their intestinal tract. Such conditions include patients receiving antineoplastic chemotherapy or radiation therapy, and those suffering antibiotic-induced colitis, and Crohn's disease. Most importantly, recipients of high dose chemotherapy followed by autologous or allogeneic hematopoietic stem cell transplant or bone marrow transplant or those diagnosed as having hematologic malignancies may require decolonization therapy during their treatment and recovery periods.

Included among therapies that make a patient high risk for developing a Gram-positive bacteremia are lengthy periods of hospitalization, especially in intensive care-units (ICUs), and high dose chemotherapy followed by autologous or allogeneic hematopoietic stem cell transplant or bone marrow transplant or solid organ transplants. Hospitalization for as little as one day, two days, or three days in an ICU can result in colonization of the intestinal tract with antibiotic-resistant Gram-positive bacteria, eventually resulting in a bacteremia caused by the colonization. Other medical therapies that result in immune system compromise include, for example, antineoplastic chemotherapy and radiation therapy, as well as the use of immunosuppressive medications. Therapies that also cause a patient to be at “high risk” for developing an antibiotic-resistant Gram-positive bacteremia include prior or concomitant antibacterial therapy using vancomycin or an antibiotic with anaerobic bacterial activity.

In patients where the elevated risk of developing a bacteremia is a result of a medical procedure or treatment (e.g., antineoplastic chemotherapy), it is preferable that antibiotic therapy to substantially decolonize the intestinal tract begin at least 1 day, 3 days, 7 days, or 14 days prior to the medical procedure or treatment. In one embodiment, decolonization proceeds concomitantly with the medical procedure. If desirable, the decolonization therapy may be continued for at least 1 day, 3 days, 7 days, or 14 days subsequent to the medical procedure.

Flora of the Intestinal Tract

Normally, in the upper gastrointestinal tract of adult humans, the esophagus contains only the bacteria swallowed with saliva and food. The acidity of the stomach contents severely limits bacterial growth. Accordingly, the proximal small intestine has relatively limited Gram-positive flora, consisting mainly of Lactobacillus spp. and Enterococcus faecalis. Typically this region has about 10⁵-10⁷ bacteria per milliliter of luminal fluid. The distal region of the small intestine contains greater numbers of Gram-positive bacteria and other normal flora including several Gram-negative species (e.g., coliforms and Bacteroides). Generally, the bacterial population and diversity increases distally, reaching 10¹¹ bacteria per gram of feces in the colon among which are Gram-positive bacterial species such as, Staphylococcus spp., Enterococcus spp., Streptococcus spp., and Clostridium spp.

Under normal conditions, the natural intestinal flora prevent colonization by pathogenic bacterial species. Additionally, the normal flora stimulate the production of cross-reactive antibodies in the host animal, acting as antigens and inducing immunological responses. Host defense mechanisms are a complex set of humoral and cellular processes that prevent microorganisms from invading the body including the bloodstream. While the normal bacterial flora are generally considered non-pathogenic in healthy individuals, these same bacteria can cause life-threatening infections if given the opportunity in patients with impaired immune function. Risk factors for these opportunistic infections include advanced age, organ transplantation, cancer, HIV infection, malnutrition, and other acquired or congenital causes of immune dysfunction as described supra. Such patients are susceptible to developing bacteremia by normal intestinal bacteria.

Likewise, disorders of the intestinal tract that compromise the barrier function of the intestinal mucosa render a patient susceptible to developing bacteremia by intestinal bacteria. Such conditions include, for example, colitis, proctitis, enteritis, mucositis, or Crohn's disease. Many of these types of conditions can be induced by therapies for other disease indications, for example, resulting from antineoplastic chemotherapy or radiotherapy, or antibiotic-induced colitis.

Traditionally, bacteremias caused by the intestinal flora were susceptible to standard antibiotic therapy, and were thus successfully treated with known conventional antibiotics. However, with the recent emergence of stains of antibiotic-resistant bacteria, treating bacteremias of this nature has become significantly more difficult. For example, VRE faecium may be resistant to all commercially-available antibiotics, including linezolid and quinupristin/dalfopristin. Furthermore, patients with underlying malignancies who are colonized by VRE have rates of VRE bacteremia as high as 19%. Patients who develop bacterermias with VRE have longer hospital and ICU stays, high mortality, and greater health care costs than patients without VRE bacteremias. Thus, identification of agents that result in the suppression and/or elimination of VRE and other intestinal antibiotic-resistant Gram-positive bacteria could significantly reduce morbidity, mortality, and cost.

The highest concentrations of antibiotic-resistant bacteria, including vancomycin-resistant Enterococcus (VRE), methicillin-resistant Staphylococcus aureus (MRSA), vancomycin-resistant Staphylococcus aureus (VRSA), glycopeptide-intermediate susceptible Staphylococcus aureus (GISA), and penicillin-resistant Streptococcus pneumoniae (PRSP), are found in hospitals, nursing homes, and other facilities where antibiotics are heavily used. Unfortunately, these same locations also have the highest density of susceptible, at-risk patients. Patient care may be improved and nosocomial infections may be reduced by preventing, rather than treating, bacteremias by decolonizing the intestinal tract of a patient identified with antibiotic-resistant bacteria.

Detection of Gram-positive Bacteria

Gram-positive bacteria that colonize the intestinal tract of a patient or cause a bacteremia can be easily detected and characterized by a skilled artisan. For example, the Gram-positive bacteria that colonize the intestinal tract can be isolated, for identification and sensitivity testing, from a stool sample, rectal swab, or culture using standard microbiological techniques. Generally, stool specimens are collected in clean (not necessarily sterile), wide-mouthed containers that can be covered with a tight-fitting lid. These containers should be free of preservatives, detergents, and metal ions and contamination with urine should also be avoided.

Stool specimens should be examined and cultured as soon as possible after collection because, as the stool specimen cools, the drop in pH soon becomes sufficient to inhibit the growth of many bacterial species. Direct microscopic examination of a fecal emulsion or stained smear to evaluate the presence of fecal pathogen forms may be valuable in the differential diagnosis of certain enteric infections. A bacterial smear for staining can also be prepared. If a delay in processing is anticipated, for example if the specimen is to be sent to a distant reference laboratory, an appropriate preservative should be used. Equal quantities of a 0.033 M sodium or potassium phosphate buffer and glycerol can be used to recover pathogenic bacteria for culturing and staining purposes.

For antibiotic sensitivity testing, a small amount of fecal specimen can be added to Gram-positive or other enrichment broth for the recovery of bacterial species. Alternatively, the broth may inoculated using a rectal swab. A variety of culture media containing inhibitors to the growth of normal bowel flora allows Gram-positive species to be selected. Subcultures of either isolated or mixed Gram-positive species can be prepared using antibiotic-containing culture media.

Alternatively, Gram-positive bacteria can be identified by molecular techniques, such as nucleic acid analyses. Some molecular techniques used in clinical microbiology for the analysis of drug-resistant bacteria have been described by Fluit et al. in Clin. Micro. Reviews 14: 836-71, 2001. A real time PCR method has been described by Grisold et al. in J. Clin. Microbiol. 40: 2392-97, 2002. Nucleic acid techniques can also be used to visualize bacteria, as described in U.S. Patent Application Serial No. 2002/0192755 A1. The above-mentioned detection techniques can be used to analyze the bacteria present in the blood or resident in the gastrointestinal tract. A comparison of blood/non-blood bacterial colonies in a patient can determine whether the prophylactic methods of the invention should be practiced.

Ramoplanin

Ramoplanin (A-16686; MDL 62,198; IB-777), a glycolipodepsipeptide antibiotic obtained from fermentation of Actinoplanes strain ATCC 33076, has activity against Gram-positive aerobic and anaerobic microorganisms. Ramoplanin consists of a major component (A2) and related minor components. Of these minor components, five have been structurally identified and designated as A1, A′1, A′2, A3, and A′3. Variations between structures A1, A2, and A3 are due to changes in the fatty acid moiety of ramoplanin; minor components A′2, A′2, and A′3 contain one fewer sugar residue.

The structure of ramoplanin is characterized by two antiparallel beta-strands, which are formed by residues 2-7 and 10-14, respectively. The beta-strands are connected by six intramolecular hydrogen bonds and a reverse beta-turn which is formed by Thr8 and Phe9. Residues 2 and 14 are connected by a loop consisting of Leu15, Ala16, Chp17, and the side chain of Asn2. Although residues 14-17 show the formation of a beta-turn, only the N-terminal end of the turn is directly connected to one of the beta-strands (Gly14), whereas the C-terminal end (Chp17) is linked via the side chain of Asn2. The 3D conformation of ramoplanin is also stabilized by a hydrophobic cluster of the aromatic side chains of the residues 3, 9, and 17. This hydrophobic collapse leads to an U-shaped topology of the beta-sheet: with the beta-turn at one end and the loop at the other end. Ramoplanin and its method of manufacture is described extensively in U.S. Pat. No. 4,303,646 (hereby incorporated by reference).

Ramoplanin inhibits the synthesis of the bacterial cell wall by inhibiting the N-acetylglucosaminyl transferase-catalyzed conversion of lipid intermediate I to lipid intermediate II, thus interfering with peptidoglycan synthesis; this mechanism is different from that of vancomycin, teicoplanin, or other cell wall-synthesis inhibitors. No evidence of cross-resistance between ramoplanin and other glycopeptides has been observed.

Ramoplanin's spectrum of activity includes staphylococci, streptococci, clostridia, enterococci, including antibiotic-resistant strains of these species (e.g., methicillin-resistant staphylococci and vancomycin- and gentamicin-resistant enterococci). Ramoplanin is bactericidal with minimal differences between the minimum inhibitory concentration (MIC) and minimum bactericidal concentration (MBC) for most Gram-positive species.

Dosages

Ramoplanin is administered orally in an amount and for a duration sufficient to substantially decolonize the intestinal tract of Gram-positive bacteria. Although the exact dosage of ramoplanin sufficient for substantially decolonizing the intestinal tract of a particular patient may differ, the dosage can be easily determined by a person of ordinary skill. Typically, the amount of ramoplanin that is administered is an amount that maintains the stool concentration of the antibiotic at least equal to the MIC for the target organism. Preferably, the amount of ramoplanin that is administered maintains the stool concentration equivalent to two, three, four, or more times the MIC for the target organism. Thus, the particular treatment regimen may vary for each patient, dependent upon the species and resistance pattern of the identified Gram-positive bacteria, and biological factors unique to each patient including the comorbidity, disease etiology, patient age (pediatric, adult, geriatric), and the nutritional and immune status.

The suggested oral dosage of ramoplanin is at least about 50, 100, 200, 300, 400, or 500 mg/day up to as much as 600, 700, 800, 900, or 1000 mg/day. An antibiotic may be given daily (e.g., once, twice, three times, or four times daily) or less frequently (e.g., once every other day, or once or twice weekly). A suitable dose is between 200 and 400 mg B.I.D. (twice daily). The antibiotic may be contained in any appropriate amount in any suitable carrier substance, and is generally present in an amount of 1-99% by weight of the total weight of the composition. The composition is provided in a dosage form that is suitable for oral administration and delivers a therapeutically effective amount of the antibiotic to the small and large intestine, as described below.

Ramoplanin is available as granules for oral solution, provided, for example, in packets containing 400 mg free base of ramoplanin, along with pharmaceutically acceptable excipients (e.g., mannitol, hydroxypropyl methylcellulose, magnesium stearate). The contents of the packet can be reconstituted with approximately 15-30 mL of water, and the resulting solution either consumed directly, or further diluted with water, cranberry juice, apple juice, or 7-Up prior to drinking. After consumption, the drug may be followed with subsequent amounts of these beverages or with food (e.g., cracker, bread). The 400 mg granulated powder packets are stable for at least one year at refrigerated conditions. The reconstituted ramoplanin aqueous solution has a shelf life of 48 hours when stored at refrigerated conditions.

The dosing regimen required to substantially decolonize the intestinal tract of Gram-positive bacteria may be altered during the course of the therapy. For example, decolonization of the intestinal tract can be monitored periodically or at regular intervals to measure the patient's bacterial load and dosage or frequency of antibiotic therapy can be adjusted accordingly.

Typically, therapy should last at least five days, but preferably at least one week, two weeks, three weeks, one month, two months, or more. The antibiotic therapy should at least encompass the period during which the patient is at highest risk for developing a bacteremia. More preferably, the antibiotic therapy should begin prior to, and extend beyond the patient's period of highest risk. For example, in the case of high dose chemotherapy followed by autologous or allogeneic hematopoietic stem cell transplant or bone marrow transplantation, antibiotic therapy should be started at least one week prior to the preparative chemotherapeutic regimen and continued until marrow engraftment has occurred and neutropenia has resolved. Preferably, antibiotic therapy continues for at least one or two weeks longer than the immunosuppressive therapy.

Pharmaceutical Formulations

Pharmaceutical compositions according to the invention may be formulated to release an antibiotic substantially immediately upon administration or at any predetermined time or time period after administration. The latter types of compositions are generally known as controlled release formulations, which include formulations that create a substantially constant concentration of the drug within the intestinal tract over an extended period of time, and formulations that have modified release characteristics based on temporal or environmental criteria.

Antibiotic-containing formulations suitable for ingestion include, for example, a pill, capsule, tablet, emulsion, solution, suspension, syrup, or soft gelatin capsule. Additionally, the pharmaceutical formulations may be designed to provide either immediate or controlled release of the antibiotic upon reaching the target site. The selection of immediate or controlled release compositions depends upon a variety of factors including the species and antibiotic susceptibility of Gram-positive bacteria being treated and the bacteriostatic/bactericidal characteristics of the therapeutics. Methods well known in the art for making formulations are found, for example, in Remington: The Science and Practice of Pharmacy (20th ed.), ed. A. R. Gennaro, 2000, Lippincott Williams & Wilkins, Philidelphia, or in Encyclopedia of Pharmaceutical Technology, eds. J. Swarbrick and J. C. Boylan, 1988-1999, Marcel Dekker, New York.

Immediate release formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, mannitol, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the antibiotic with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice. Other useful controlled release compositions are known in the art (see, for example, U.S. Pat. Nos. 4,946,685 and 6,261,601).

Formulations which target ramoplanin release to particular regions of the intestinal tract can also be prepared. Ramoplanin can be encapsulated in an enteric coating which prevents release degradation and release from occurring in the stomach, but dissolves readily in the mildly acidic or neutral pH environment of the small intestine. A formulation targeted for release of antibiotic to the colon, utilizing technologies such as time-dependent, pH-dependent, or enzymatic erosion of polymer matrix or coating can also be used.

Alternatively, a multilayer formulation having different release characteristics between the layers can be prepared. These formulations can result in the antibiotic being released in different regions of the intestinal tract. A multilayer formulation of this type may be particularly useful for maintaining a more constant antibiotic concentration throughout the length of the intestinal tract.

The targeted delivery properties of the ramoplanin-containing formulation may be modified by other means. For example, the antibiotic may be complexed by inclusion, ionic association, hydrogen bonding, hydrophobic bonding, or covalent bonding. In addition polymers or complexes susceptible to enzymatic or microbial lysis may also be used as a means to deliver drug.

Microsphere encapsulation of ramoplanin is another useful pharmaceutical formulation for targeted antibiotic release. The antibiotic-containing microspheres can be used alone for antibiotic delivery, or as one component of a two-stage release formulation. Suitable staged release formulations may consist of acid stable microspheres, encapsulating ramoplanin to be released later in the lower intestinal tract admixed with an immediate release formulation to deliver antibiotic to the stomach and upper duodenum.

Microspheres can be made by any appropriate method, or from any pharmaceutically acceptable material. Particularly useful are proteinoid microspheres (see, for example, U.S. Pat. Nos. 5,601,846, or 5,792,451) and PLGA-containing microspheres (see, for example, U.S. Pat. Nos. 6,235,224 or 5,672,659). Other polymers commonly used in the formation of microspheres include, for example, poly-ε-caprolactone, poly(ε-caprolactone-Co-DL-lactic acid), poly(DL-lactic acid), poly(DL-lactic acid-Co-glycolic acid) and poly(ε-caprolactone-Co-glycolic acid) (see, for example, Pitt et al., J. Pharm. Sci., 68:1534, 1979). Microspheres can be made by procedures well known in the art including spray drying, coacervation, and emulsification (see for example Davis et al. Microsphere and Drug Therapy, 1984, Elsevier; Benoit et al. Biodegradable Microspheres: Advances in Production Technologies, Chapter 3, ed. Benita, S, 1996, Dekker, New York; Microencapsulation and Related Drug Processes, Ed. Deasy, 1984, Dekker, New York; U.S. Pat. No. 6,365,187).

Liquids for Oral Administration

Powders, dispersible powders, or granules suitable for preparation of aqueous solutions or suspensions by addition of water are convenient dosage forms for oral administration. Formulation as a suspension provides the active ingredient in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

### EXAMPLE 1

### Suppression of VRE in a Mouse Model

Mice were colonized with a clinical isolate VanA strain of E. faecium (VRE) isolated from a septicemic patient. A single inoculation of 5×10⁸ cfu VRE by oral gavage (Day 0) was followed by treatment with vancomycin in the drinking water to maintain colonization. On day 22, each group received the same vancomycin-containing drinking water. One group also received ramoplanin (100 μg/mL) in its drinking water. The dose of ramoplanin per day was estimated to be 15 mg/kg, based on a standard water consumption of 150 mL/kg/day. Treatment with ramoplanin was discontinued on Day 29, and vancomycin treatment was discontinued on Day 36. The control group consisted of five mice, while the ramoplanin group consisted of four mice.

Treatment with ramoplanin significantly reduced the fecal density and carriage of VRE in mice. After one week of treatment, the VRE concentration per gram of feces fell from 9.7 log units to an undetectable level (<3.1 log units) in all animals. Seven days after treatment with ramoplanin, the VRE concentration per gram of feces was similar to the pre-treatment levels. The results are shown in Table 1.

TABLE 1

| | | | | Enterococci | |
|---|---|---|---|---|---|
| | | | | (log 10 cfu/g | |
| | | | | faeces | |
| | | | % Mice | | Total |
| Day | Study Phase | Treatment | with VRE | VRE | Enterococci |
| 22 | Prior to | 25 mg/kg/day | 100 | 9.7 | 9.6 |
| | ramoplanin | vancomycin | | | |
| | therapy | (control) | | | |
| | | 25 mg/kg/day | 100 | 9.7 | 9.8 |
| | | vancomycin | | | |
| 29 | Completion of | 25 mg/kg/day | 100 | 9.4 | 9.3 |
| | ramoplanin | vancomycin | | | |
| | therapy | (control) | | | |
| | | 25 mg/kg/day | 0 | <3.1 | <2.4 |
| | | vancomycin + | | | |
| | | 15 mg/kg/day | | | |
| | | ramoplanin | | | |
| 36 | 7 days after | 25 mg/kg/day | 100 | 9.3 | 9.6 |
| | completion of | vancomycin | | | |
| | ramoplanin | (control) | | | |
| | therapy | 25 mg/kg/day | 100 | 8.7 | 8.6 |
| | | vancomycin | | | |

### EXAMPLE 2

### Oral Bioavailability of Ramoplanin

The oral availability of ramoplanin was assessed by comparing a 1000 mg/kg oral dose with a 5 mg/kg intravenous dose in the rat. The absolute bioavailability was very poor (F=0.18% percent bioavailability relative to intravenous administration). The mean absorption time was 3.4 hours. Maximum observed serum concentrations were 11.6 g/mL at 0.083 hours following the 5 mg/kg intravenous dose, and 2.8 μg/mL at 0.5 hours following the 1000 mg/kg oral dose. Terminal elimination half-lives were 3.5 and 5.9 hours for the intravenous and oral doses, respectively. The volume of the distribution at steady state was 0.75 L/kg and the volume of the central compartment was 0.42 L/kg, indicating that ramoplanin is not widely distributed outside the central compartment. In intravenously treated rats, the amount excreted in urine represented less than 0.01% of the administered dose.

The bioavailability of a 300 mg/kg ramoplanin oral gelatin capsule and the serum pharmacokinetics of a 5 mg/kg intravenous dose were examined in four male Beagle dogs. The absolute bioavailability was less than 1%, and the mean absorption time was 8.19±3.12 hours. Ramoplanin was not distributed widely to tissues.

### EXAMPLE 3

### Oral Administration of Ramoplanin to Humans

As is described in detail below, single oral doses (up to 1000 mg) and multiple oral doses (200, 400, or 800 mg B.I.D. for 10 days) of ramoplanin have been administered to healthy male volunteers. Both bioassay and HPLC-based assays to assess the absorption, distribution, metabolism, and excretion were utilized in these studies. Ramoplanin was not detected in serum/plasma or urine by either method, indicating that very little, if any, is absorbed. Treatment with oral ramoplanin at all doses was efficacious in reducing the Gram-positive colony counts in feces to undetectable levels during the 10-day regimen. Ramoplanin was not effective against Gram-negative flora.

Single Dose Study in Healthy Male Volunteers

The absorption, tolerability, and recovery of ramoplanin following single dose oral administration were investigated in male volunteers. Ramoplanin was administered as an aqueous solution at a dose of 100, 200, 500, or 1000 mg to fasting subjects. Serum samples were obtained prior to drug administration of ramoplanin and 0.5, 1, 2, 3, 6, 9, 12, 24, 48, 72, and 96 hours after treatment. Urine samples were collected prior to administration of ramoplanin and over the periods 0-3, 3-6, 6-12, 12-24, 24-48, 48-72, and 72-96 hours after dosing. Fecal samples were collected prior to dosing and over the periods 0-16 (Day 1), 16-40 (Day 2), 40-64 (Day 3), 64-88 (Day 4), and 88-96 (Day 5) hours after dosing. A microbiological assay employing Bacillis subtilis ATCC 6633 as the test organism was used to determine ramoplanin concentrations in serum, urine, and feces. The limits of quantitation for this assay were 0.02 μg/mL in serum, 0.012 μg/mL in urine, and 3 μg/g in feces. Tolerability was assessed on the basis of clinical signs and symptoms and the results of blood and urine laboratory tests.

Ramoplanin concentrations in feces varied widely due to the variation in the weight of the fecal samples (6-468 g); detectable concentrations ranged from 2.9 to 278 μg/g in the 100 mg group, 7.7 to 454 μg/g in the 200 mg group, 6.6 to 3316 μg/g in the 500 mg group, and 16.0 to 3154 μg/g in the 1000 mg group. Maximum ramoplanin concentrations in feces, as well as maximum percentage recoveries, generally occurred the day after administration (Day 2). The time of occurrence of maximum ramoplanin concentrations in feces was not dose dependent. In contrast, the maximum ramoplanin fecal concentrations were dose-dependent. Mean maximum concentrations were 214 μg/g (range 148-278 μg/g), 287 μg/g (range 164-454 μg/g), 1655 μg/g (range 737-3316 μg/g), and 1835 μg/g (range 1336-3154 μg/g) for the 100, 200, 500, and 1000 mg groups, respectively. Mean cumulative recovery of ramoplanin in feces for the 100, 200, 500, and 1000 mg groups were 67.7% (range 55.7-84%), 48.5% (range 39.3-56.5%), 52.8% (range 41.3-79.6%), and 46.4% (range 39.9-58.4%) of the administered dose, respectively. On the fourth day of study, ramoplanin was still detectable in feces obtained from 17 of 24 subjects.

Multiple Dose Study in Healthy Male Volunteers

Healthy male volunteers were administered 200, 400, or 800 mg ramoplanin twice-a-day, for ten consecutive days. The predetermined dose was reconstituted in 5 mL water per vial, mixed with 50 mL of sweetened, aromatized solution, and immediately administered orally to the subjects.

No absorption from the human gastrointestinal tract was observed. On Days 1, 5, and 10, no serum levels of ramoplanin were detected at hour 0.5, 1, 2, 3, 6, 9, and 12 after the morning dose. No levels were found in urine at Day 1 and 5, or in the pooled urine samples of the periods 0-12, 12-24, 24-36, 48-72, and 72-96 after the last dose.

The fecal concentrations of ramoplanin were dose related on both Day 3 (average concentration 827, 1742, 1901 μg/g in the 200, 400, and 800 mg group, respectively) and Day 10 (949, 1417, 2647 μg/g, respectively). The concentrations declined on the first day post-treatment, but remained detectable in some subjects four days post-treatment. The cumulative recovery up to Day 4 post-treatment was 25% of the administered dose.

The antibacterial activity of ramoplanin on the stool microflora was assessed in a subset of the subjects. Microbial concentrations (i.e., the number of organisms per gram of fecal matter) were determined at the following time points: Day-4 (pre-treatment), Days 4 and 10 (treatment), and Days 7 and 24 (follow-up). Tolerability and absorption were also investigated.

As expected, no effect was seen in Gram-negative bacteria (enteric bacteria and Bacteroides spp.) or yeast. A marked effect was seen on Gram-positive bacteria by the first measurement on Day 4. In all subjects, the concentrations of staphylococci, streptococci, and enterococci were below the level of detection by Day 10. In 10 of 12 subjects, the concentration of ramoplanin and vancomycin-resistant Clostridium spp. was reduced below detectable levels. In the other two subjects who carried ramoplanin- and vancomycin-resistant Clostridium spp. (C. rectum and C. beijerinckii) before treatment, no variation in the clostridial load was observed. No ramoplanin- or vancomycin-resistant strain of C. difficile was detected, either pre- or post-treatment.

After therapy, the intestinal tracts of the volunteers were re-colonized by normal Gram-positive bacteria, with a tendency for enterococci and clostridia to transiently achieve concentrations higher than the basal level. To evaluate if the predominant species that colonized the intestinal tract after therapy was that isolated before treatment, all enterococci isolated before and after ramoplanin therapy were speciated using the API system. DNA-typing was also performed when identification at the strain level was necessary. In most cases, the predominant appeared to be different before and after treatment, suggesting a lack of persistence of the initial isolate.

The in vitro interaction of ramoplanin with human intestinal contents was studied. Ramoplanin was found to be microbiologically active in feces and to bind reversibly to solid components of feces. The binding and the subsequent release of ramoplanin from feces would likely result in long-lasting concentrations in the intestinal tract.

Multiple Dose Study in Asymptomatic Carriers of Intestinal VRE

Patients identified as asymptomatic carriers of VRE were administered placebo or one of two dosages (100 mg, 400 mg) of ramoplanin b.i.d. (twice daily) for seven days. Patients were assessed by rectal swab on Days 7, 14, and 21 to determine the presence or absence of VRE. On Days 45 and 90, stool samples were analyzed for long-term effects of ramoplanin on the recurrence of, or re-infection with, VRE. All VRE isolates were tested for susceptibility to ramoplanin.

Analysis of the primary efficacy variable showed that ramoplanin effectively suppressed intestinal VRE (i.e., ramoplanin substantially decolonized the intestinal tract of VRE). None of the placebo-treated patients were VRE-free after seven days of treatment. In contrast, 17 of 21 patients (81.0%; p<0.01) who received 100 mg ramoplanin b.i.d. and 18 of 20 patients (90.0%; p<0.01) who received 400 mg ramoplanin b.i.d. were had no detectable VRE at Day 7. Seven days after cessation of treatment (Day 14), 6 of 21 patients (28.6%) who received 100 mg ramoplanin b.i.d. and 7 of 17 patients (41.2%) who received 400 mg ramoplanin b.i.d. remained VRE free. At Day 21, the number of VRE-free patients was comparable among all treatment groups.

### EXAMPLE 4

### Comparison of the Pharmacodynamic Effects of Different Oral Formulations of Ramoplanin in Humans

To further elucidate the pharmacodynamics and intestinal bioavailability, healthy male volunteers (18-45 years; BMI—19-29 kg/m²) were administered oral ramoplanin (400 mg b.i.d.) for seven days and fecal Enterococci were monitored. Ramoplanin was administered to six subjects as a granulated powder admixed with Orasweet® syrup vehicle.

The presence of fecal enterococci was confirmed in all subjects prior to study initiation. Fecal samples were cultured on day 7/8. The pharmacodynamic endpoints was the number of subjects with successful suppression of fecal Enterococci on day 7/8 compared to the pre-study culture.

The growth of fecal enterococci was suppressed in all test subjects. The fecal ramoplanin concentration was 0.56-2.65 mg/g. No measurable ramoplanin concentration was detected in the plasma or urine samples of any subject indicating that ramoplanin was not absorbed from the gastrointestinal tract.

### EXAMPLE 5

### Bactericidal Activity of Ramoplanin Against Linezolid-resistant and Quinupristin/dalfopristin-resistant VRE

The efficacy of ramoplanin was tested against recently isolated VRE that are resistant to linezolid and quinupristin/dalfopristin. The culture media containing ramoplanin was supplemented with 0.02% bovine serum albumin (BSA) to prevent ramoplanin binding to the plastic. MIC values were determined in both cation-adjusted Mueller-Hinton broth (CAMHB) and trypticase soy broth (TSB) by the National Committee for Clinical Laboratory Standards (NCCLS) broth microdilution method. Macromolecular biosynthesis experiments were done by following the incorporation of carbon-14-labeled N-acetylglucosamine, acetic acid, amino acids, uridine, and thymidine into peptidoglycan, fatty acid, protein, RNA and DNA, respectively.

The MIC of ramoplanin for all VRE cultured in CAMHB in this study was 0.125-0.25 μg/ml. Time-kill studies demonstrate the bactericidal activity of ramoplanin against VRE. Macromolecular biosynthesis experiments demonstrate that peptidoglycan synthesis was the first macromolecular biosynthetic pathway inhibited by ramoplanin at the MIC.

Table 2 shows the MIC (μg/ml) or ramoplanin against 29 strains of bacteria.

TABLE 2

| | Ramoplanin | Ramoplanin | Vancomycin | Vamcomycin |
|---|---|---|---|---|
| Strain | CAMHB | TSB | CAMHB | TSB |
| E. faecium | 0.25, 0.25 | 0.5, 0.5 | >16, >16 | >16, >16 |
| A2735 Syn-R | | | | |
| E. faecium | 0.25, 0.25 | 0.5, 0.5 | >16, >16 | >16, >16 |
| A4192 Syn-R | | | | |
| E. faecium | 0.25, 0.25 | 0.25, 0.25 | 2, 2 | 2, 2 |
| A6343 Line-R | | | | |
| E. faecium | 0.125, 0.125 | 0.25, 0.25 | >16, >16 | >16, >16 |
| A6345 Line-R | | | | |
| E. faecium | 0.25, 0.25 | 0.25, 0.25 | >16, >16 | >16, >16 |
| A 6350 Line-R | | | | |
| E. faecium | 0.25, 0.25 | 0.5, 0.5 | 2, 2 | 2, 2 |
| A6350 Line-R | | | | |
| E. faecium | 0.125, 0.125 | 0.25, 0.25 | >16, >16 | >16, >16 |
| A5959 Line-R | | | | |
| E. faecium | 0.125, 0.125 | 0.25, 0.25 | >16, >16 | >16, >16 |
| A-5960 Line-R | | | | |
| E. faecalis | 0.125, 0.125 | 0.25, 0.25 | 2, 2 | 2, 2 |
| A7789 Line-R | | | | |
| E. faecium | 0.125, 0.125 | 0.125, 0.125 | >16, >16 | >16, >16 |
| UA210 | | | | |
| E. faecium | 0.25, 0.25 | 0.25, 0.25 | 1, 1 | 2, 2 |
| UA392 | | | | |
| E. gallinarum | 0.125, 0.125 | 0.25, 0.25 | 1, 1 | 2, 2 |
| UA604 | | | | |
| E. faecium | 0.5, 0.5 | 0.5, 0.5 | >16, >16 | >16, >16 |
| 1836 | | | | |
| E. faecalis | 0.5, 0.5 | 0.5, 0.5 | 4, 4 | >16, >16 |
| ATCC29212 | | | | |
| E. faecalis | 0.5, 0.5 | 0.5, 0.5 | 4, 4 | 2, 2 |
| ATCC29212 | | | | |
| E. faecalis | 0.5, 0.5 | 0.5, 0.5 | >16, >16 | >16, >16 |
| ATCC51299 | | | | |
| E. faecium | 0.25, 0.25 | 0.5, 0.5 | >16, >16 | >16, >16 |
| ATCC700221 | | | | |
| S. epidermidis | 0.5, 0.5 | 1, 1 | 4, 4 | 4, 4 |
| ATCC12228 | | | | |
| S. epidermidis | 0.5, 0.5 | 0.5, 0.5 | 4, 4 | 4, 4 |
| ATCC14990 | | | | |
| S. epidermidis | 0.5, 0.5 | 1, 1 | 4, 4 | 4, 4 |
| ATCC18972 | | | | |
| S. epidermidis | 0.5, 0.5 | 2, 1 | 4, 4 | 4, 4 |
| ATCC35983 | | | | |
| S. epidermidis | 0.5, 0.5 | 0.5, 0.5 | 4, 4 | 4, 4 |
| ATCC35984 | | | | |
| S. aureus | 0.5, 0.5 | 0.5, 0.5 | 2, 2 | 2, 2 |
| ATCC12600 | | | | |
| MRSA | | | | |
| S. aureus | 0.5, 0.5 | 0.25, 0.25 | 2, 2 | >16, >16 |
| ATCC19636 | | | | |
| MSSA | | | | |
| S. aureus | 0.25, 0.25 | 0.25, 0.25 | 1, 1 | >16, >16 |
| ATCC27659 | | | | |
| MSSA | | | | |
| S. aureus | 0.5, 1 | 0.25, 0.25 | 2, 2 | 2, 2 |
| ATCC27660 | | | | |
| MSSA | | | | |
| S. aureus | 0.5, 0.5 | 0.125, 0.125 | 1, 1 | >16, >16 |
| ATCC35556 | | | | |
| MRSA | | | | |
| S. aureus | 1, 1 | 0.5, 0.5 | 2, 2 | 2, 2 |
| ATCC43300 | | | | |
| MRSA | | | | |
| S. aureus | 0.5, 0.25 | 0.25, 0.25 | 2, 2 | 2, 2 |
| ATCC700699 | | | | |
| MRSA/VISA | | | | |

“Line-R” indicates a linezolid-resistant strain.
“Syn-R” indicates a quinupristin/dalfopristin (Synercid ®)-resistant strain.

Table 3 shows the reference MIC (μg/ml) for strains used for killing curve.

TABLE 3

| Strain | Ramoplanin | Vancomycin | Linezolid | Chloramphenicol | Rifampin |
|---|---|---|---|---|---|
| E. faecium | 0.25 | >64 | 16-32 | 16 | 16 |
| A6345 | | | | | |
| Line-R. | | | | | |
| E. faecium | 0.25 | >64 | 16 | 16 | 0.03 |
| A6345 | | | | | |
| Line-R. | | | | | |

“Line-R.” indicates a linezolid-resistant strain.

Table 4 shows the killing curve for E. faecium A6345.

TABLE 4

| Time | | | | | | | |
|---|---|---|---|---|---|---|---|
| Point | | Chlor. | Chlor. | Rif. | Rif. | Ramo. | Ramo. |
| (hrs) | Control | 4XMIC | 1XMIC | 4XMIC | 1XMIC | 4XMIC | 1XMIC |
| 0 | 400000 | 400000 | 400000 | 400000 | 400000 | 400000 | 400000 |
| | (5.6020) | (5.6020) | (5.6020) | (5.6020) | (5.6020) | (5.6020) | (5.6020) |
| 2 | 1500000 | 380000 | 370000 | 350000 | 350000 | 340000 | 390000 |
| | (6.1760) | (5.5797) | (5.5682) | (5.5440) | (5.5440) | (5.5314) | (5.5910) |
| 4 | 5700000 | 330000 | 320000 | 310000 | 320000 | 190000 | 310000 |
| | (6.7558) | (5.5185) | (5.5051) | (5.4913) | (5.5051) | (5.2787) | (5.4913) |
| 6 | 14000000 | 350000 | 350000 | 330000 | 340000 | 150000 | 250000 |
| | (7.1461) | (5.5440) | (5.5440) | (5.5185) | (5.5314) | (5.1760) | (5.3979) |
| 24 | 270000000 | 110000 | 470000 | 930000 | 2500000 | 0 | 140 |
| | (8.4313) | (5.6720) | (5.6720) | (5.9684) | (6.3979) | (0.00) | (2.1461) |

Data is presented as cfu/ml.
Log₁₀(cfu/ml) is provided in parenthesis.
Chlor. = chloramphenicol;
Rif. = rifamycin;
Ramo. = ramoplanin.

Table 5 shows the killing curve for E. faecium A6349.

TABLE 5

| Time | | | | | | | |
|---|---|---|---|---|---|---|---|
| Point | | Chlor. | Chlor. | Rif. | Rif. | Ramo. | Ramo. |
| (hrs) | Control | 4XMIC | 1XMIC | 4XMIC | 1XMIC | 4XMIC | 1XMIC |
| 0 | 500000 | 500000 | 500000 | 500000 | 500000 | 500000 | 500000 |
| | (5.6989) | (5.6989) | (5.6989) | (5.6989) | (5.6989) | (5.6989) | (5.6989) |
| 2 | 3000000 | 500000 | 530000 | 470000 | 490000 | 390000 | 490000 |
| | (6.4771) | (5.6989) | (5.7242) | (5.6720) | (5.6901) | (5.5910) | (5.6901) |
| 4 | 10000000 | 420000 | 490000 | 450000 | 420000 | 190000 | 400000 |
| | (7.00) | (5.6232) | (5.6901) | (5.6532) | (5.6232) | (5.2787) | (5.6020) |
| 6 | 35000000 | 460000 | 500000 | 400000 | 410000 | 90000 | 270000 |
| | (7.5440) | (5.6627) | (5.6989) | (5.6020) | (5.6127) | (4.9542) | (5.4313) |
| 24 | 200000000 | 400000 | 800000 | 260000 | 210000 | 0 | 200 |
| | (8.3010) | (5.6020) | (5.9030) | (5.4149) | (5.3222) | (0.00) | (2.3010) |

Data is presented as cfu/ml.
Log₁₀(cfu/ml) is provided in parenthesis.
Chlor. = chloramphenicol;
Rif. = rifamycin;
Ramo. = ramoplanin.

Table 6 shows ramoplanin, vancomycin, methicillin, and linezolid MIC (μg/ml) against 30 strains of bacteria.

TABLE 6

| | Ramo. | Ramo. | Vanco. | Vanco. | Meth. | Meth. | Line. | Line. |
|---|---|---|---|---|---|---|---|---|
| Strain | 1^{st} | 2^{nd} | 1^{st} | 2^{nd} | 1^{st} | 2^{nd} | 1^{st} | 2^{nd} |
| E. faecium | 0.25 | 0.25 | >16 | >64 | >16 | >64 | 4 | 2 |
| A2735 | | | | | | | | |
| Syn-R | | | | | | | | |
| E. faecium | 0.25 | 0.5 | >16 | >64 | >16 | >64 | 4 | 2 |
| A4192 | | | | | | | | |
| Syn-R | | | | | | | | |
| E. faecium | 0.125 | 0.25 | 1 | 1 | >16 | 64 | 8 | 8 |
| A6343 | | | | | | | | |
| Line-R | | | | | | | | |
| E. faecium | 0.125 | 0.25 | >16 | >64 | >16 | >64 | >16 | 32 |
| A6345 | | | | | | | | |
| Line-R | | | | | | | | |
| E. faecium | 0.125 | 0.25 | >16 | >64 | >16 | >64 | 16 | 16 |
| A6349 | | | | | | | | |
| Line-R | | | | | | | | |
| E. faecium | 0.25 | 0.25 | 1 | 1 | >16 | 64 | 4 | 2 |
| A6350 | | | | | | | | |
| Line-R | | | | | | | | |
| E. faecium | 0.125 | 0.25 | >16 | >64 | >16 | >64 | >16 | 16 |
| A5959 | | | | | | | | |
| Line-R | | | | | | | | |
| E. faecium | 0.125 | 0.25 | >16 | >64 | >16 | >64 | >16 | 32 |
| A-5960 | | | | | | | | |
| Line-R | | | | | | | | |
| E. faecalis | 0.06 | 0.125 | 1 | 1 | >16 | 32 | >16 | 32 |
| A7789 | | | | | | | | |
| Line-R | | | | | | | | |
| E. faecium | 0.06 | 0.125 | >16 | >64 | >16 | >64 | 4 | 2 |
| UA210 | | | | | | | | |
| E. faecium | 0.125 | 0.25 | 2 | 2 | >16 | >64 | 4 | 2 |
| UA392 | | | | | | | | |
| E. gallinarum | 0.06 | 0.125 | 0.5 | 0.5 | >16 | >64 | 4 | 2 |
| UA604 | | | | | | | | |
| E. faecium | 0.25 | 0.5 | >16 | >64 | >16 | >64 | 4 | 2 |
| 1836 | | | | | | | | |
| E. faecalis | 0.25 | 0.5 | 2 | 2 | >16 | 32 | 4 | 2 |
| ATCC29212 | | | | | | | | |
| E. faecalis | 0.25 | 0.25 | 2 | 2 | 16 | 32 | 4 | 2 |
| ATCC29212 | | | | | | | | |
| E. faecalis | 0.25 | 0.5 | >16 | 32 | >16 | 32 | 2 | 2 |
| ATCC51299 | | | | | | | | |
| E. faecium | 0.25 | 0.25 | >16 | >64 | >16 | >64 | 4 | 2 |
| ATCC700221 | | | | | | | | |
| S. epidermidis | 1 | 0.25 | 2 | 2 | 1 | 2 | 2 | 2 |
| ATCC12228 | | | | | | | | |
| S. epidermidis | 0.5 | 0.25 | 2 | 2 | 1 | 1 | 4 | 2 |
| ATCC14990 | | | | | | | | |
| S. epidermidis | 1 | 0.25 | 2 | 2 | >16 | 64 | 2 | 1 |
| ATCC18972 | | | | | | | | |
| S. epidermidis | 1-0.5 | 0.5 | 2 | 2 | >16 | 64 | 2 | 2 |
| ATCC35983 | | | | | | | | |
| S. epidermidis | 1 | 0.25 | 2 | 2 | >16 | >64 | 4 | 2 |
| ATCC35984 | | | | | | | | |
| S. aureus | 1 | 0.5 | 1 | 1 | 2 | >64 | 4 | 2 |
| ATCC12600 | | | | | | | | |
| MRSA | | | | | | | | |
| S. aureus | 1 | 1 | 1 | 1 | 1 | 2 | 4 | 2 |
| ATCC19636 | | | | | | | | |
| MSSA | | | | | | | | |
| S. aureus | 1 | 0.25 | 1 | 1 | 2 | 2 | 4 | 2 |
| ATCC27659 | | | | | | | | |
| MSSA | | | | | | | | |
| S. aureus | 2 | 1 | 1 | 1 | 2 | 4 | 4 | 2 |
| ATCC27660 | | | | | | | | |
| MSSA | | | | | | | | |
| S. aureus | 1 | 0.5 | 1 | 1 | 1 | 1 | 4 | 2 |
| ATCC35556 | | | | | | | | |
| MRSA | | | | | | | | |
| S. aureus | 2 | 0.25 | 1 | 1 | 8 | 8 | 4 | 2 |
| ATCC43300 | | | | | | | | |
| MRSA/VISA | | | | | | | | |
| S. aureus | 1 | 0.5 | 2 | 2 | >16 | >64 | 4 | 2 |
| ATCC700699 | | | | | | | | |
| MRSA/VISA | | | | | | | | |

“Line-R” and “Syn-R” aere as defined above

### EXAMPLE 6

### Combination Therapy for Treating Patients Diagnosed as Having a VRE Bacteremia

Frequently, patients develop a VRE bacteremia before effective intestinal decolonization therapy can be administered. In such cases, decolonization therapy using ramoplanin may be combined with traditional anti-VRE therapy. For example, an institutionalized patient diagnosed as having a VRE bacteremia may be treated with a combination of oral ramoplanin (400 mg b.i.d.) and high dose linezolid (600 mg intravenously every 12 hours). Alternatively, because linezolid is absorbed from the gastrointestinal tract, oral linezolid dosing may be appropriate (see, for example, Linden, Drugs 62: 425-441, 2002).

Linezolid treatment continues until the systemic bacteremia has been eradicate, but usually for no less than 14 days. Decolonization therapy using oral ramoplanin continues for at least the same duration as linezolid therapy, but preferably for an additional period of up to 3, 7, or 14 days. Ramoplanin decolonization therapy may be of shorter duration than linezolid therapy if clinically indicated.

Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

8. The method of claim 1, wherein said high risk patient is diagnosed as having an autoimmune disorder.

9. The method of claim 8, wherein said autoimmune disorder is systemic lupus erythematosus, rheumatoid arthritis, scleroderma, dermatomyositis/polymyositis, Sjogren's syndrome, mixed connective tissue disorders, Behcet's syndrome, sarcoidosis, or vasculitides.

10. The method of claim 1, wherein said high risk patient is receiving immunosuppressant therapy.

11. The method of claim 10, wherein said immunosuppressant therapy comprises a corticosteroid, an anti-thymocyte globulin, cyclosporin, or tacrolimus.

12. The method of claim 1, wherein said high risk patient is diagnosed having increased intestinal permeability.

13. The method of claim 1, wherein said high risk patient is diagnosed as having or at risk for developing enteritis, colitis, or mucositis of the intestinal tract.

14. The method of claim 1, wherein said high risk patient is diagnosed as having an illness requiring hospitalization or institutionalization for at least five consecutive days.

15. The method of claim 1, wherein said high risk patient is diagnosed as having an illness requiring hospitalization in an intensive care unit for at least three consecutive days.

16. The method of claim 1, wherein said high risk patient is admitted to a hospital or healthcare institution in which antibiotic-resistant Gram-positive bacteria are endemic.

17. The method of claim 16, wherein said Gram-positive bacteria are VRE, MRSA, or VRSA.

18. The method of claim 1, wherein said Gram-positive bacteria are antibiotic-resistant.

19. The method of claim 18, wherein said antibiotic-resistant Gram-positive bacteria comprise bacteria of the genus Enterococcus.

20. The method of claim 19, wherein said bacteria are E. faecium, E. faecalis, E. raffinosus, E. avium, E. hirae, E. gallinarum, E. casseliflavus, E. durans, E. malodoratus, E. mundtii, E. solitarius, or E. pseudoavium.

21. The method of claim 20, wherein said bacteria are resistant to vancomycin.

22. The method of claim 20, wherein said bacteria are resistant to one or more antibiotics selected from the group consisting of teicoplanin, daptomycin, oritavancin, dalbavancin, everninomycin, quinupristin/dalfopristin, linezolid, and tigecycline.

23. The method of claim 20, wherein said bacteria are resistant to one or more antibiotics selected from the group consisting of glycopeptides, everninomycins, streptogramins, lipopeptides, oxazolidonones, bacteriocins, type A lantibiotics, type B lantibiotics, liposidomycins, mureidomycins, and alkanoylcholines,

24. The method of claim 18, wherein said antibiotic-resistant Gram-positive bacteria comprise bacteria of the genus Staphylococcus.

25. The method of claim 24, wherein said bacteria are S. aureus, S. epidermidis, S. hominis, S. saprophyticus, S. hemolyticus, S. capitis, S. auricularis, S. lugdenis, S. warneri, S. saccharolyticus, S. caprae, S. pasteurii, S. schleiferi, S. xylosus, S. cohnii, or S. simulans.

26. The method of claim 25, wherein said bacteria are resistant to methicillin.

27. The method of claim 24, wherein said bacteria are resistant to one or more antibiotics selected from the group consisting of teicoplanin, daptomycin, oritavancin, dalbavancin, eveminomycin, quinupristin/dalfopristin, linezolid, and tigecycline.

28. The method of claim 24, wherein said bacteria are resistant to one or more antibiotics selected from the group consisting of glycopeptides, eveminomycins, streptogramins, lipopeptides, oxazolidonones, bacteriocins, type A lantibiotics, type B lantibiotics, liposidomycins, mureidomycins, and alkanoylcholines,

29. The method of claim 18, wherein said antibiotic-resistant Gram-positive bacteria comprise bacteria of the genus Streptococcus.

30. The method of claim 29, wherein said bacteria are S. pyogenes, S. agalactiae, S. pneumoniae, S. bovis, S. aureus, or a member of the viridans group of streptococci.

31. The method of claim 29, wherein said bacteria are resistant to penicillin.

32. The method of claim 29, wherein said bacteria are resistant to one or more antibiotics selected from the group consisting of teicoplanin, daptomycin, oritavancin, dalbavancin, everninomycin, quinupristin/dalfopristin, linezolid, and tigecycline.

33. The method of claim 29, wherein said bacteria are resistant to one or more antibiotics selected from the group consisting of glycopeptides, everninomycins, streptogramins, lipopeptides, oxazolidonones, bacteriocins, type A lantibiotics, type B lantibiotics, liposidomycins, mureidomycins, and alkanoylcholines.

34. The method of claim 1, wherein said ramoplanin is formulated such that substantially all of said ramoplanin is non-absorbable or partially non-absorbable, and retains antibacterial activity in the lumen of the intestinal tract of said patient.

35. The method of claim 1, wherein said ramoplanin is administered twice daily at a dosage of between about 100 mg and 800 mg.

36. The method of claim 35, wherein said ramoplanin is administered twice daily at a dosage of between about 200 mg and 400 mg.

37. The method of claim 35, wherein said Gram-positive bacteria is vancomycin-resistant Enterococcus.

38. The method of claim 35, wherein said Gram-positive bacteria is a methicillin-resistant Staphylococcus or vancomycin-resistant Staphylococcus aureus (VRSA).

39. The method of claim 35, wherein said Gram-positive bacteria is resistant to linezolid or quinupristin/dalfopristin.

40. The method of claim 35, wherein said ramoplanin is administered for at least 7 days.

41. The method of claim 40, wherein said ramoplanin is administered for at least 14 days.

42. A method for treating a bacteremia in a patient, wherein said bacteremia is caused by Gram-positive bacteria, comprising administering to said patient:
(a) a bioavailable antibiotic in an amount and duration sufficient to treat said bacteremia; and
(b) oral ramoplanin in an amount and for a duration sufficient to substantially decolonize the intestinal tract of said patient of said Gram-positive bacteria.

43. The method of claim 42, wherein said bioavailable antibiotic is selected from the group consisting of almecillin, amdinocillin, amikacin, amoxicillin, amphomycin, amphotericin B, ampicillin, azacitidine, azaserine, azithromycin, azlocillin, aztreonam, bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mupirocin, mycophenolic acid, nafcillin, natamycin, neomycin, netilmicin, niphimycin, nisin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, Pep5, RP 59500, and TD-6424.

44. The method of claim 42, wherein said bioavailable antibiotic is a member of one of the antibiotic families selected from the group consisting of bacteriocins, type A lantibiotics, type B lantibiotics, liposidomycins, mureidomycins, alanoylcholines, quinolines, eveminomycins, glycylcyclines, carbapenems, cephalosporins, streptogramins, oxazolidonones, tetracyclines, cyclothialidines, bioxalomycins, cationic peptides, and protegrins.

45. The method of claim 42, wherein said Gram-positive bacteria are antibiotic-resistant.

46. The method of claim 45, wherein said antibiotic-resistant Gram-positive bacteria comprise bacteria of the genus Enterococcus.

47. The method of claim 46, wherein said bacteria are E. faecium, E. faecalis, E. raffinosus, E. avium, E. hirae, E. gallinarum, E. casseliflavus, E. durans, E. malodoratus, E. mundtii, E. solitarius, or E. pseudoavium.

48. The method of claim 46, wherein said bacteria are resistant to vancomycin.

49. The method of claim 46, wherein said bacteria are resistant to one or more antibiotics selected from the group consisting of teicoplanin, daptomycin, oritavancin, dalbavancin, everninomycin, quinupristin/dalfopristin, linezolid, and tigecycline.

50. The method of claim 46, wherein said bacteria are resistant to one or more antibiotics selected from the group consisting of glycopeptides, everninomycins, streptogramins, lipopeptides, oxazolidonones, bacteriocins, type A lantibiotics, type B lantibiotics, liposidomycins, mureidomycins, and alkanoylcholines,

51. The method of claim 45, wherein said antibiotic-resistant Gram-positive bacteria comprise bacteria of the genus Staphylococcus.

52. The method of claim 51, wherein said bacteria are S. aureus, S. epidermidis, S. hominis, S. saprophyticus, S. hemolyticus, S. capitis, S. auricularis, S. lugdenis, S. warneri, S. saccharolyticus, S. caprae, S. pasteurii, S. schleiferi, S. xylosus, S. cohnii, or S. simulans.

53. The method of claim 51, wherein said bacteria are resistant to methicillin.

54. The method of claim 51, wherein said bacteria are resistant to one or more antibiotics selected from the group consisting of teicoplanin, daptomycin, oritavancin, dalbavancin, everninomycin, quinupristin/dalfopristin, linezolid, and tigecycline.

55. The method of claim 51, wherein said bacteria are resistant to one or more antibiotics selected from the group consisting of glycopeptides, everninomycins, streptogramins, lipopeptides, oxazolidonones, bacteriocins, type A lantibiotics, type B lantibiotics, liposidomycins, mureidomycins, and alkanoylcholines,

56. The method of claim 45, wherein said antibiotic-resistant Gram-positive bacteria comprise bacteria of the genus Streptococcus.

57. The method of claim 56, wherein said bacteria are S. pyogenes, S. agalactiae, S. pneumoniae, S. bovis, S. aureus, or a member of the viridans group of streptococci.

58. The method of claim 56, wherein said bacteria are resistant to penicillin.

59. The method of claim 56, wherein said bacteria are resistant to one or more antibiotics selected from the group consisting of teicoplanin, daptomycin, oritavancin, dalbavancin, everninomycin, quinupristin/dalfopristin, linezolid, and tigecycline.

60. The method of claim 56, wherein said bacteria are resistant to one or more antibiotics selected from the group consisting of glycopeptides, everninomycins, streptogramins, lipopeptides, oxazolidonones, bacteriocins, type A lantibiotics, type B lantibiotics, liposidomycins, mureidomycins, and alkanoylcholines.

61. The method of claim 42, wherein said ramoplanin is formulated such that substantially all of said ramoplanin is non-absorbable or partially non-absorbable, and retains antibacterial activity in the lumen of the intestinal tract of said patient.

62. The method of claim 42, wherein said ramoplanin is administered twice daily at a dosage of between about 100 mg and 800 mg.

63. The method of claim 62, wherein said ramoplanin is administered twice daily at a dosage of between about 200 mg and 400 mg.

64. The method of claim 62, wherein said Gram-positive bacteria is vancomycin-resistant Enterococcus.

65. The method of claim 62, wherein said Gram-positive bacteria is a methicillin-resistant Staphylococcus or vancomycin-resistant Staphylococcus aureus (VRSA).

66. The method of claim 62, wherein said Gram-positive bacteria is resistant to linezolid or quinupristin/dalfopristin.

67. The method of claim 62, wherein said ramoplanin is administered for at least 7 days.

68. The method of claim 67, wherein said ramoplanin is administered for at least 14 days.
